(19) United States
(12) Patent Application Publication
(10) Pub. No.: US 2013/0269568 A1
(43) Pub. Date: Oct. 17, 2013

(54) DERIVATIVES FOR PERFLUOROALKOXY SULFOSUCCINATES AS SURFACTANTS

(71) Applicants: Eckhard Claus, Frankfurt am Main (DE); Melanie Kleineidam, Darmstadt (DE); Wolfgang Hierse, Gross-Zimmern (DE)

(72) Inventors: Eckhard Claus, Frankfurt am Main (DE); Melanie Kleineidam, Darmstadt (DE); Wolfgang Hierse, Gross-Zimmern (DE)

(73) Assignee: MERCK PATENT GmbH, Darmstadt (DE)

(21) Appl. No.: 13/995,233

(22) PCT Filed: Nov. 26, 2011
(86) PCT No.: PCT/EP11/05952

§ 371 Date: Jun. 18, 2013

(30) Foreign Application Priority Data
Dec. 21, 2010 (EP) 10015855.9

(51) Int. Cl. C07C 59/315; C09D 11/00; C09D 7/12

(52) U.S. Cl. CPC C07C 59/315; C09D 7/1233; C09D 11/00
USPC 106/31.13; 560/151; 106/287.24

(57) ABSTRACT

The present invention relates to novel compounds containing Rf end groups, to the use thereof as surface-active substances, and to processes for the preparation of these compounds. The claimed compounds fall under the following formula: (I), Two examples of claimed compound are: (II), (III).

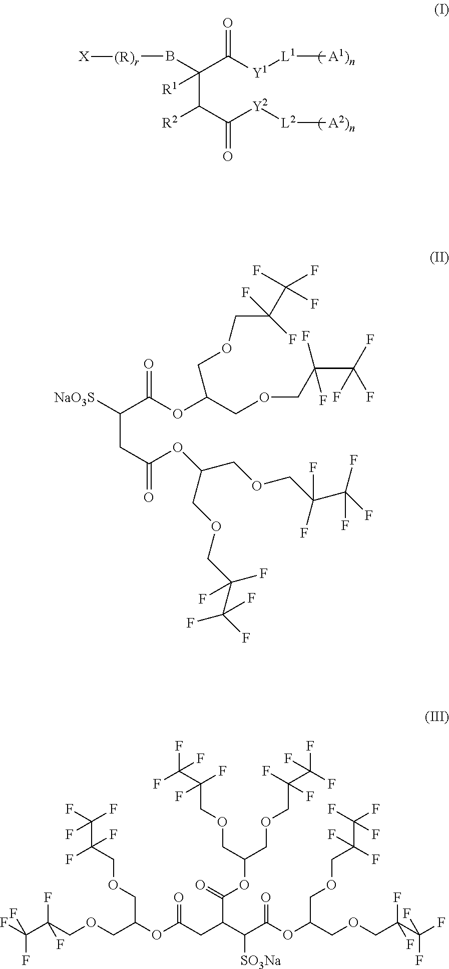

The present invention relates to novel compounds containing fluorinated end groups, to the use thereof as surface-active substances, and to processes for the preparation of these compounds.

Fluorosurfactants have an outstanding ability to reduce surface tension, which is utilised, for example, in the hydrophobicisation of surfaces, for example of textiles, paper, glass, building materials or adsorbents. In addition, it is possible to use them as interface promoter or emulsifier or viscosity reducer in paints, surface coatings or adhesives.

In general, fluorosurfactants contain perfluoroalkyl substituents, which are degraded to perfluoroalkylcarboxylic acids (PFCAs) and -sulfonic acids (PFASs) in the environment by biological and/or other oxidation processes. In recent years, the accumulation of perfluoroalkylcarboxylic acids (PFCAs) and perfluoroalkylsulfonic acids (PFASs) in nature has given cause for concern. PFCAs and PFASs are highly persistent compounds whose long-chain variants (containing perfluoroalkyl chains of 8 or more carbon atoms) have a bioaccumulative potential. They are in some cases suspected of causing health problems (G. L. Kennedy, Jr., J. L. Butenhoff, G. W. Olsen, J. C. O'Connor, A. M. Seacat, R. G. Biegel, S. R. Murphy, D. G. Farrar, Critical Review in Toxicology, 2004, 34, 351-384).

Sulfosuccinates and/or sulfotricarballylates containing various fluorinated side chains are described in U.S. Pat. No. 4,968,599, U.S. Pat. No. 4,988,610 and U.S. Pat. No. 6,890,608 and in A. R. Pitt et al., Colloids and Surfaces A: Physicochemical and Engineering Aspects, 1996, 114, 321-335; A. R. Pitt, Progr. Colloid Polym. Sci., 1997, 103, 307-317 and Z.-T. Liu et al., Ind. Eng. Chem. Res. 2007, 46, 22-28. The Omnova company markets polymers whose side chains contain terminal CF₃ or C₂F₅ groups. International Patent Application WO 03/010128 describes perfluoroalkyl-substituted amines, acids, amino acids and thioether acids which contain a C3-20-perfluoroalkyl group. JP-A-2001/133984 discloses surface-active compounds containing perfluoroalkoxy chains which are suitable for use in antireflection coatings. JP-A-09/111,286 discloses the use of perfluoropolyether surfactants in emulsions. International patent application WO 2006/072401 describes compounds which carry at least one terminal pentafluorosulfuranyl group or at least one terminal trifluoromethoxy group and contain a polar end group, are surface-active and are suitable as surfactants.

There continues to be a demand for alternative surface-active substances, preferably having a property profile comparable to that of classical fluorosurfactants and equally great chemical versatility, which are preferably not degraded to long-chain persistent fluorocarboxylic or fluorosulfonic acids on oxidative or reductive degradation or are preferably effective as conventional fluorosurfactants in relatively low dosage.

Novel compounds have now been found which are suitable as surface-active substances and preferably do not have one or more of the above-mentioned disadvantages. The novel surface-active compounds can be used as individual components or, if advantageous, also in mixtures.

The present invention relates firstly to compounds of the formula (I)

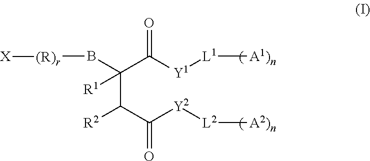

where
X is a hydrophilic group,
R is linear or branched alkylene, where one or more non-adjacent C atoms may be replaced by O, S, and/or N,
r is 0 or 1,
B is a single bond, O, NH, NR′, CH₂, C(O)—O, S, CH₂—O, O—C(O), O—C(O)—O, N—C(O), C(O)—N, O—C(O)—N, N—C(O)—N, SiR′₂—, SiR′₂—O, O—SO₂ or SO₂—O,
where R′ is linear or branched alkyl,
R¹ and R² are, independently of one another, hydrogen or —CH₂—COY³-L³-(A³)ₙ₃,
Y¹, Y² and Y³ are, independently of one another, O, S or N,
L¹, L² and L³ are, independently of one another, linear or branched alkylene, where one or more non-adjacent C atoms may be replaced by O, S, and/or N,
A¹, A² and A³ are, independently of one another, hydrogen or a group of the structure —Zⁱ(CR³R⁴)ₘᵢRfⁱ,

### Zⁱ is O, S or N,

R³ and R⁴ are, independently of one another, hydrogen or an alkyl group,
Rfⁱ is a fluorine-containing radical,
n1, n2 and n3 are, independently of one another, 1-6,
m1, m2 and m3 are, independently of one another, 0-5
and the compounds contain at least one Rfⁱ group.

Preferred compounds of the formula (I) are those in which R¹ and R² are not simultaneously —CH₂—COY³-L³-(A³)ₙ₃.

The compounds according to the invention may contain one or more Rfⁱ groups. A¹, A² and A³ are preferably, independently of one another, a group of the structure —Zⁱ(CR³R⁴)ₘᵢRfⁱ. n1, n2 and n3 are preferably not simultaneously equal to 1. n1, n2 and n3 are particularly preferably, independently of one another, 2-3. Particular preference is given to compounds containing at least four Rfⁱ groups. A preferred variant are compounds containing four, six or nine Rfⁱ groups.

Fluorinated groups Rfⁱ which can be used are branched or unbranched, fluorine-containing alkyl radicals or CF₃O groups.

The fluorinated groups Rfⁱ used are preferably branched or unbranched, fluorine-containing alkyl radicals, in particular perfluorinated alkyl radicals. Preference is furthermore given to fluorine-containing alkyl radicals having 1 to 10, preferably 1 to 6, in particular 1 to 4 C atoms. Preference is given to the use of perfluorinated Rfⁱ groups having 1 to 6, in particular 1 to 4 C atoms. Rf¹, Rf² and Rf³ preferably have the same meaning.

In another variant of the invention, CF₃O groups can preferably be used, in particular if Y is S or N.

The groups Rfⁱ which are essential to the invention are bonded to a group L¹, L² or L³ via a group —Zⁱ(CR³R⁴)ₘᵢ. Zⁱ here preferably stands for O or N, in particular for O. Preference is given to compounds in which all Zⁱ are identical. R³ and R⁴ preferably stand, independently of one another, for hydrogen or an unbranched C1-C3-alkyl group. m1, m2 and m3 preferably stand, independently of one another, for 1-3. Preference is given to compounds in which all Zⁱ, R³, R⁴ and mi in each case have the same meaning.

L¹, L² and L³ can preferably, independently of one another, be linear or branched alkylene having 1 to 10 C atoms. In particular, L¹, L² and L³ are, independently of one another, linear or branched alkylene having 3 to 8 C atoms. One or more non-adjacent C atoms of the groups L¹, L² and L³ may preferably be replaced by O or N, preferably by O. In a preferred variant of the invention, L¹ and L² are identical. If L³ is also present, L¹ and L² or L¹ and L³ or L² and L³ may preferably be identical. In a particularly preferred variant of the invention, all groups L¹, L² and L³ are identical.

In an embodiment of the invention, the compounds according to the invention may be present in the form of mixtures, in which the individual compounds have different meanings for Lⁱ, Aⁱ, Zⁱ, Rfⁱ, ni and mi, in particular for ni and mi.

In the compounds according to the invention, Y¹, Y² and Y³ are preferably O or N, in particular O. Y¹, Y² and Y³ preferably have the same meaning.

In the compounds according to the invention, X is a hydrophilic group, preferably an anionic, cationic, nonionic or amphoteric group.

A preferred anionic group X can be selected from —COO⁻, —SO₃⁻, —OSO₃⁻, —PO₃²⁻, —OPO₃²⁻, —(OCH₂CH₂)ₛ—O—(CH₂)ₜ—COO⁻, —(OCH₂CH₂)ₛ—O—(CH₂)ₜ—SO₃²⁻, —(OCH₂CH₂)ₛ—O—(CH₂)ₜ—OSO₃⁻, —(OCH₂CH₂)ₛ—O—(CH₂)ₜ—PO₃²⁻, —(OCH₂CH₂)ₛ—O—(CH₂)ₜ—OPO₃²⁻ or for the formulae A to C,

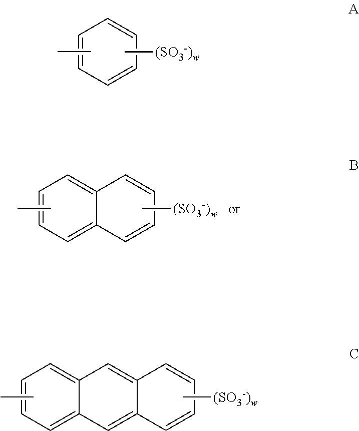

where s stands for an integer from the range 1 to 1000, t stands for an integer selected from 1, 2, 3 or 4, and w stands for an integer selected from 1, 2 or 3.

The preferred anionic groups here include, in particular, —COO⁻, —SO₃⁻, —OSO₃⁻, —PO₃²⁻, —OPO₃²⁻, the sub-formula A, and —(OCH₂CH₂)ₛ—O—(CH₂)ₜ—COO⁻, —(OCH₂CH₂)ₛ—O—(CH₂)ₜ—SO₃⁻ and —(OCH₂CH₂)ₛ—O—(CH₂)ₜ—OSO₃⁻, where each individual one of these groups taken alone may be preferred. The very particularly preferred anionic groups here include —SO₃⁻, —OSO₃⁻, —PO₃²⁻ or OPO₃²⁻, in particular —SO₃⁻. Particular preference is given to a sulfonate group —SO₃⁻.

The preferred counterion for anionic groups X is a monovalent cation, in particular H⁺, an alkali metal cation or NR₄⁺, where R=H or C1-C6-alkyl, and all R may be identical or different. Particular preference is given to Na⁺, K⁺ or NH₄⁺, especially preferably Na⁺.

A preferred cationic group X can be selected from —NR¹R²R³⁺Z⁻, —PR¹R²R³⁺Z⁻,

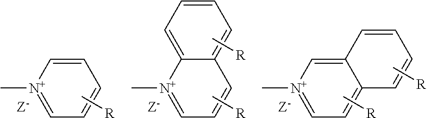

where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻
R¹, R² and R³ each stand, independently of one another, for H, C₁₋₃₀-alkyl, Ar or —CH₂Ar and
Ar stands for an unsubstituted or mono- or polysubstituted aromatic ring or condensed ring systems having 6 to 18 C atoms in which, in addition, one or two CH groups may be replaced by N.

The preferred cationic groups here include, in particular, from —NR¹R²R³⁺Z and

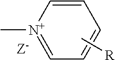

where each individual one of these groups may be preferred per se.

A preferred nonionic group can be selected from linear or branched alkyl, where one or more non-adjacent C atoms may be replaced by O, S, and/or N, —OH, —SH, —O-(glycoside)ₒ, —S-(glycoside)ₒ, —OCH₂—CHOH—CH₂—OH, —OCH₂Ar(-NCO)ₚ, —OAr(—NCO)ₚ, —CR═CH₂, —OCOCR═CH₂,

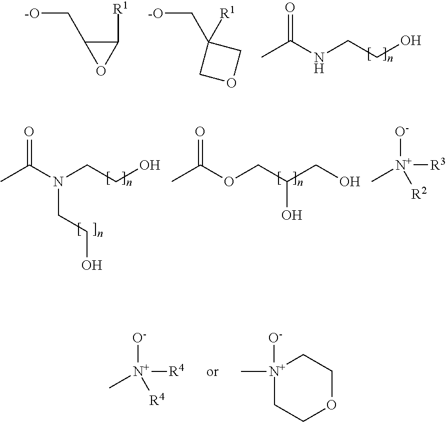

n an integer from the range from 1 to 6, preferably 1 to 4
o stands for an integer from the range from 1 to 10,
p stands for 1 or 2,
R¹, R² and R³ each stand, independently of one another, for C₁₋₃₀-alkyl, Ar or —CH₂Ar, preferably C₁₋₂₀-alkyl,
R⁴ stands for C₁₋₄-alkyl-OH, and,
Ar stands for an unsubstituted, mono- or polysubstituted aromatic ring or condensed ring systems having 6 to 18 C atoms in which, in addition, one or two CH groups may be replaced by C═O and,
glycoside stands for an etherified carbohydrate, preferably for a mono- di-, tri- or oligoglucoside,
and R stands for H or methyl.

The preferred nonionic groups here include, in particular, linear or branched alkyl, where one or more non-adjacent C atoms may be replaced by O, S and/or N, —OH, —OCOCR═CH₂ and —O-(glycoside)ₒ.

If X=alkyl, it is then preferably equal to R-(B-A)ₘ- where R=H or C₁₋₄-alkyl, in particular H or CH₃, A=linear or branched alkylene, preferably having 1 to 10 carbon atoms, in particular having 1 to 4 carbon atoms, B=O or S, preferably O, and m=an integer preferably from the range from 1 to 100, particularly preferably 1 to 30.

The nonionic group X is particularly preferably the group R—(O—CH₂CHR)ₘ— where m=an integer from the range from 1 to 100, preferably 1 to 30, and R=H or C₁₋₄-alkyl, in particular H or CH₃. R-(B-A)ₘ- is particularly preferably a polyethylene or polypropylene glycol unit.

A preferred amphoteric group can be selected from the functional groups of the acetyldiamines, the N.-alkylamino acids, the betaines, the amine oxides or corresponding derivatives, in particular selected from:

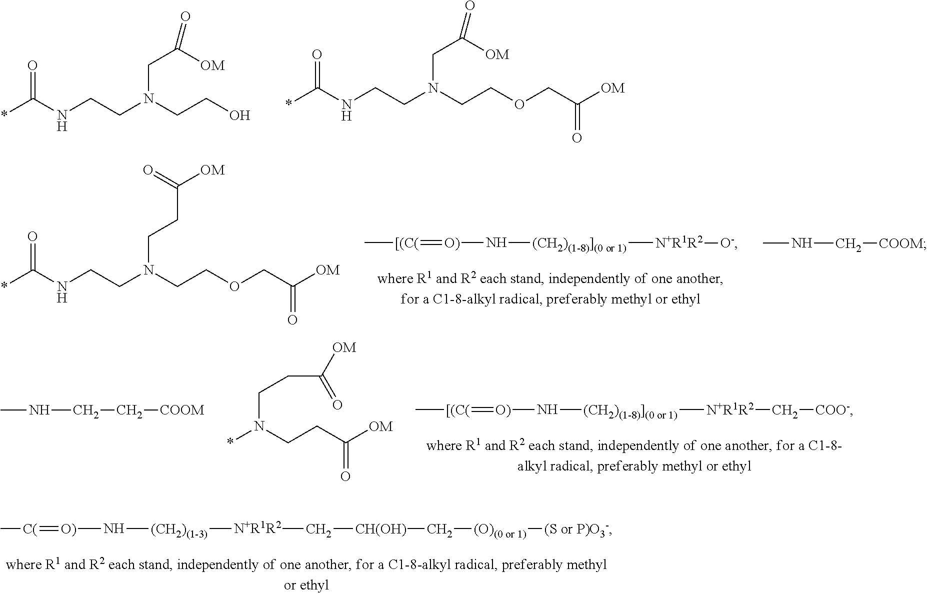

Particularly preferred compounds according to the invention are those which contain an anionic group X. Particular preference is given to compounds which contain the groups —SO₃⁻, —OSO₃⁻, —PO₃²⁻ or OPO₃²⁻, in particular —SO₃⁻. Preferred counterions here are Na⁺, K⁺ and NH₄⁺, in particular Na⁺.

The group R preferably stands for linear or branched alkylene, preferably having 1 to 12 carbon atoms, in particular having 1 to 4 carbon atoms. One more non-adjacent C atoms may preferably be replaced by O or S, preferably O.

In the compounds according to the invention, r can preferably be equal to 0.

In the compounds according to the invention, B stands for a single bond, O, NH, NR′, CH₂, C(O)—O, S, CH₂—O, O—C(O), O—C(O)—O, N—C(O), C(O)—N, O—C(O)—N, N—C(O)—N, SiR′₂—, SiR′₂—O, O—SO₂ or SO₂—O, where R′ is linear or branched alkyl. B is preferably a single bond, O, S, C(O)—O or O—C(O), in particular a single bond.

Preferred compounds are, in particular, those compounds in which all variables have the preferred meanings.

Particular preference is given to compounds in which all groups A are equal to the same group —Zⁱ(CR³R⁴)^{mi}Rfⁱ, where Rfⁱ=perfluorinated alkyl radicals having 1 to 6, in particular 1 to 4 C atoms, Z=O, R³=R⁴=hydrogen or an unbranched C1-C3-alkyl group, mi=1-3, all L=linear or branched alkylene having 3 to 8 C atoms and X=SO₃⁻, r=0 and B=a single bond.

The compounds according to the invention are preferably based on esters of hydroxysuccinic acid and of citric acid, where the compounds contain at least one Rfⁱ group.

In a preferred group of compounds of the formula I, R¹ and R² stand for hydrogen and A¹ and A² stand for a —Zⁱ(CR³R⁴)ₘᵢRfⁱ group. These compounds are represented by formula (II). Particular preference is given to compounds of the formula (II) where Y¹, Y², Z¹ and Z² are equal to O.

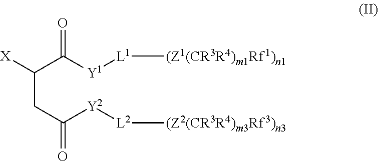

In another preferred group of compounds of the formula I, R¹ stands for H, R² stands for —CH₂—COY³-L³-(A³)ₙ₃ and A¹, A² and A³ stand for a —Zⁱ(CR³R⁴)ₘᵢRfⁱ group. These compounds are represented by formula (III). Particular preference is given to compounds of the formula (III) where Y¹, Y², Y³, Z¹, Z² and Z³ are equal to O.

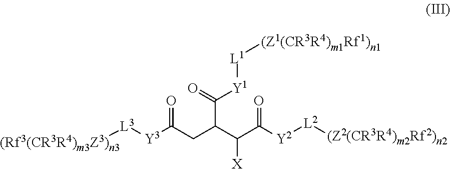

In a further preferred group of compounds of the formula I, R¹ stands for —CH₂—COY³-L³-(A³)ₙ₃, R₂ stands for hydrogen and A¹, A² and A³ stand for a —Zⁱ(CR³R⁴)ₘᵢRfⁱ group. These compounds are represented by formula (IV). Particular preference is given to compounds of the formula (IV) where Y¹, Y², Y³, Z¹, Z² and Z³ are equal to O.

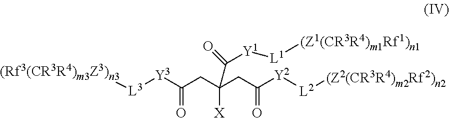

A preferred variant of the invention are X-functionalised succinates which contain at least four Rf groups and X-functionalised tricarballylates which contain at least six Rf groups. Particular preference is given to succinates containing four and citric acid esters containing six Rf groups.

Particularly preferred compounds according to the invention are compounds of the formulae (II), (III) and (IV) in which X is an anionic group. Particular preference is given to compounds of the formulae (II), (III) and (IV) which contain the groups —SO₃⁻, —OSO₃⁻, —PO₃²⁻ or OPO₃²⁻, in particular —SO₃⁻. Preferred counterions here are Na⁺, K⁺ and NH₄⁺, in particular Na⁺.

Particularly preferred compounds according to the invention are also those in which X is a polyethylene or polypropylene glycol unit, in particular a polyethylene glycol unit.

The compounds of the formulae (I) to (IV) according to the invention may also be in the form of isomer mixtures (constitutional and/or configurational isomer mixtures). In particular, diastereomer and/or enantiomer mixtures are possible.

In the formulae (II), (III) and (IV), L¹, L² and L³ have the general and preferred meanings given for the formula (I). L¹, L² and L³ are preferably, independently of one another, equal to linear or branched C1-C10-alkylene, in particular linear or branched C3-C8-alkylene. L¹ and L² are particularly preferably, independently of one another, equal to linear or branched C5-C10-alkylene for compounds of the formula (II). For compounds of the formula (III) and (IV), L¹, L² and L³ are preferably, independently of one another, equal to linear or branched C3-C6-alkylene. Particular preference is given to compounds of the formulae (II), (III) and (IV) in which all L are identical.

Particular preference is given to compounds in which all variables have the preferred meanings.

Examples of particularly preferred compounds according to the invention are compounds of the formulae (V) and (VI) in which the variables have the general and preferred meanings given for the formula (I) and yi and zi, independently of one another, be equal to 1-10:

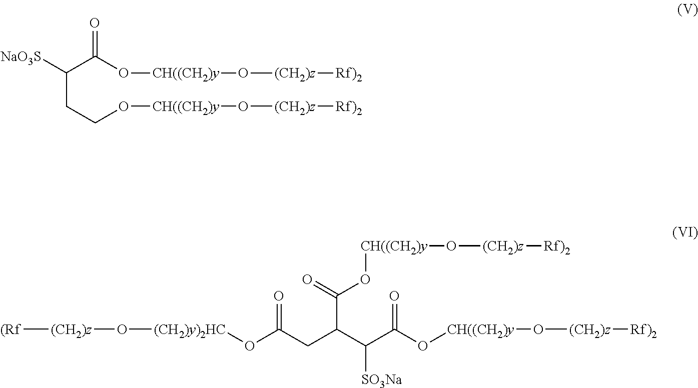

In the formulae (V) and (VI), Rfⁱ are preferably fluorine-containing alkyl radicals having 1 to 6, in particular 1 to 4 C atoms. Preference is given to the use of perfluorinated Rfⁱ groups having 1 to 4 C atoms. In the compounds of the formulae (V) and (VI), yi and zi can, independently of one another, be equal to 1-10, preferably equal to 1-6, in particular 1-3.

Particular preference is given to compounds of the formula (V) and (VI) in which perfluorinated Rfⁱ groups having 1 to 4 C atoms, in particular having 1-3 C atoms are used and in which yi and zi are, independently of one another, equal to 1-3, in particular equal to 1. Especial preference is given to compounds in which all Rfⁱ, all yi and all zi are identical.

Advantages of the compounds according to the invention may be, in particular: a surface activity which is equal or superior to that of conventional hydrocarbon surfactants with respect to efficiency and/or effectiveness, biological and/or abiotic degradability of the substances without the formation of persistent perfluorinated degradation products, such as PFOA (perfluorooctanoic acid) or PFOS (perfluorooctanesulfonic acid), weak foam formation, good processability in formulations and/or storage stability. The compounds according to the invention preferably have particular surface activity.

The present invention relates secondly to the use of at least one compound of the formula (I) as surface-active agents, for example for improving the flow behaviour and wetting ability of coating formulations.

Preference is given to the use of compounds of the formulae (II) to (IV), in particular those of the formulae (V) and (VI). The preferred embodiments of the compounds according to the invention described above can particularly advantageously be used here. Succinates which contain at least four, in particular four, Rfⁱ groups and tricarballylates which contain at least six, in particular six, Rfⁱ groups are preferably used. The compounds according to the invention can be employed individually or as a mixture of two or more compounds according to the invention. The compounds of the formulae (I) to (VI) according to the invention can also be used as isomer mixtures (constitutional and/or configurational isomer mixtures). In particular, diastereomer and/or enantiomer mixtures are possible.

Areas of application are, for example, the use of the compounds according to the invention as additives in surface-coating preparations, such as paints, coatings, protective coatings, speciality coatings in electronic or semiconductor applications (for example photoresists, top antireflective coatings, bottom antireflective coatings) or in optical applications (for example photographic coatings, coatings of optical elements) or in additive preparations for addition to corresponding preparations.

For use, the compounds according to the invention are usually incorporated into correspondingly designed compositions. The present invention likewise relates to corresponding preparations comprising at least one compound according to the invention. Such compositions preferably comprise a vehicle which is suitable for the particular application and optionally further active substances and/or optionally assistants. Preferred compositions here are paint and surface-coating preparations and printing inks.

In addition, the present invention also relates to water-based surface-coating formulations which comprise at least one of the compounds according to the invention, alone or mixed with other surfactants. Preference is given to the use of surface-coating formulations based on the following synthetic film formers: polycondensation resins, such as alkyd resins, saturated/unsaturated polyesters, polyamides/imides, silicone resins; phenolic resins; urea resins and melamine resins, polyaddition resins, such as polyurethanes and epoxy resins, polymerisation resins, such as polyolefins, polyvinyl compounds and polyacrylates.

In addition, the compounds according to the invention are also suitable for use in surface coatings based on natural products and modified natural products. Preference is given to surface coatings based on oils, polysaccharides, such as starch and cellulose, and also based on natural resins, such as cyclic oligoterpenes, polyterpenes and/or shellac.

The compounds according to the invention can be used both in physically hardening (thermoplastics) and in crosslinking (elastomers and thermosets) aqueous surface-coating systems. The compounds according to the invention preferably improve the flow and wetting properties of the surface-coating systems.

The present invention relates to all uses mentioned here of compounds to be employed in accordance with the invention. The respective use of surfactants for the said purposes is known to the person skilled in the art, and consequently the use of the compounds to be employed in accordance with the invention presents no problems.

The present invention relates thirdly to a process for the preparation of compounds of the formula (I). The compounds according to the invention can be prepared here by methods known per se to the person skilled in the art from the literature.

The compounds according to the invention can preferably be prepared by esterification of maleic acid and aconitic acid or anhydrides or acid chlorides thereof using one or more alcohols of the formula (VII)

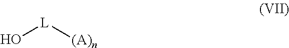

and subsequent addition onto the double bond in order to introduce the group X—(R)ᵣ-B. The compounds according to the invention can also preferably be prepared by esterification of hydroxysuccinic acid and citric acid using one or more alcohols of the formula (VII) and subsequent functionalisation of the hydroxyl groups in order to introduce the group X—(R)ᵣ-B

L and A in the formula (VII) and in the following formulae (VIII) to (XI) have the meaning described for L¹, L² and L³ or A¹, A² and A³ respectively in formula (I), in particular also the preferred meanings. The alcohols of the formula (VII) may contain two or more Rf groups, preferably two Rf groups.

The alcohols used are commercially available and/or their preparation is familiar to the person skilled in the art (for example Carbohydrate Research 1991, 219, 33).

Succinates and tricarballylates according to the invention are preferably synthesised in a two-step synthesis via the corresponding maleates or hydroxysuccinates or the corresponding aconitic or citric acid esters, which are prepared in the presence of a conventional catalyst, such as, for example, toluene-4-sulfonic acid monohydrate:

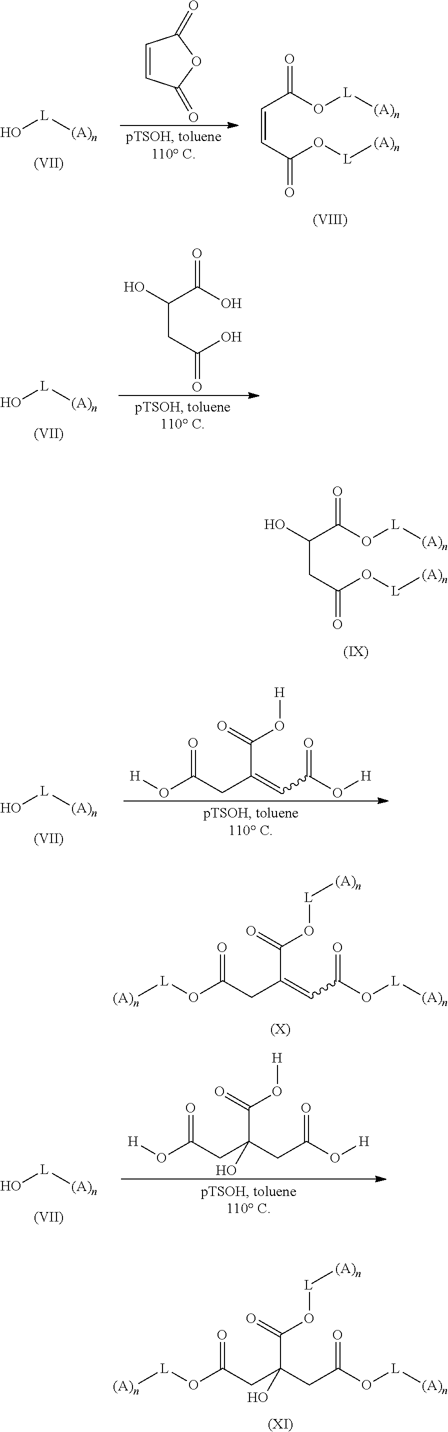

In the second step, the group X—(R)ᵣ-B is then introduced by addition onto the double bond or derivatisation of the OH group by methods familiar to the person skilled in the art.

Formula (X) shows the presence of Z/E double-bond isomers. The preparation of further compounds according to the invention can be carried out analogously to the illustrative reactions shown above. The preparation of further compounds according to the invention can also be carried out by other methods known per se to the person skilled in the art from the literature. In particular, other esterification catalysts can be used.

Furthermore, the compounds of the formula (I) can be synthesised starting from citric acid. Further possibilities for the synthesis of nonionic surfactants from citric/aconitic acid are shown by way of example in the following schemes.

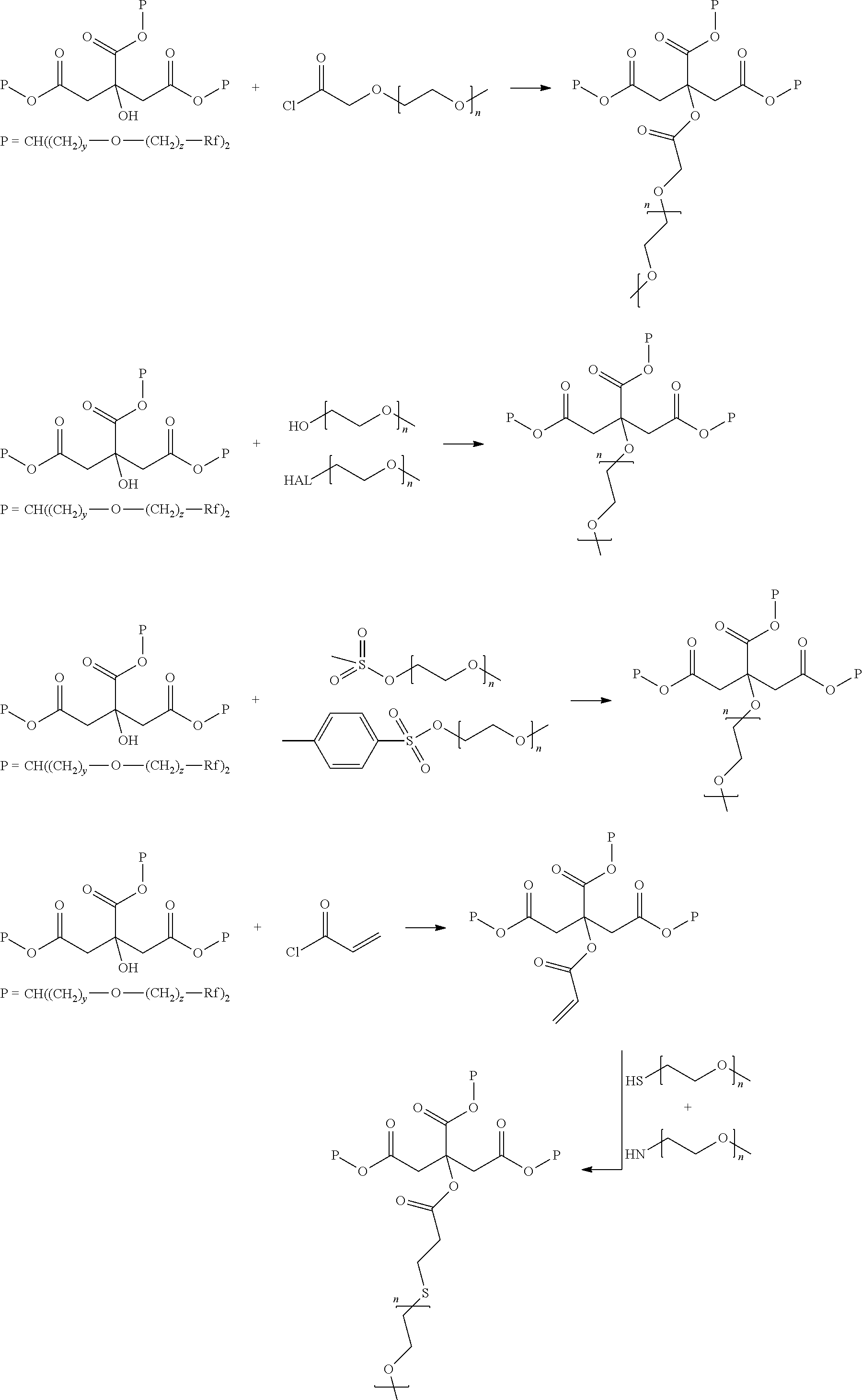

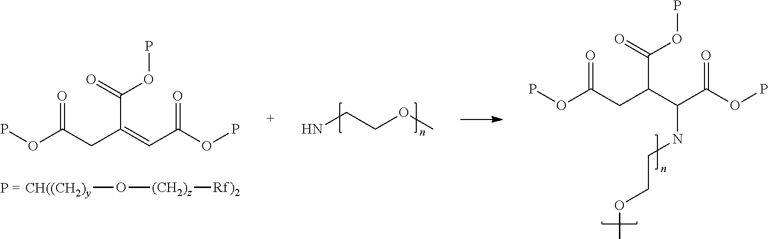

In all reactions, n is preferably 1-30.

The disclosures in the references cited hereby expressly also belong to the disclosure content of the present application. The following examples explain the present invention in greater detail without restricting the scope of protection.

### EXAMPLES

### Abbreviations

PEG: polyethylene glycol
DCM: dichloromethane
RT: room temperature
h: hour
MTB ether: methyl tert-butyl ether

### N,N-DMF: N,N-dimethylformamide

DI: deionised water
eq: equivalent

### Example 1

### Synthesis of the C₂F₅-Functionalised Sulfosuccinate of the Formula (IA)

### a) Synthesis of the Branched Alcohol

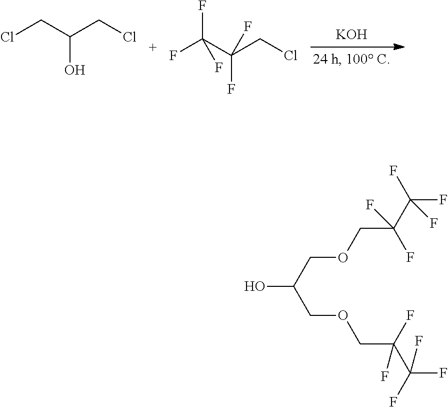

A mixture of 1,3-dichloro-2-propanol, 3 eq of 1H,1H-pentafluoropropanol and 3 eq of potassium hydroxide is heated at 100° C. for 24 h. The mixture is subsequently cooled to room temperature, and DI water and MTB ether are added, and the phases are separated. The aqueous phase is extracted with MTB ether, and the combined organic phases are washed with water, dried over sodium sulfate and filtered. The solvent is distilled off in a rotary evaporator.

Substance: C₉H₁₀F₁₀O₃; M=356.158 g/mol

¹H-NMR (400 MHz; DMSO-d₆) δ=4.14 (t, 4H); 3.81 (t, 1H); 3.68-3.49 (m, 4H) ppm.

¹⁹F-NMR (376 MHz; DMSO-d₆) δ=−83.48-−83.61 (m, 6F); −123.36-−123.53 (m, 4F) ppm.

GC-MS

[M]=64.547%

### b) Preparation of the Maleic Acid Ester

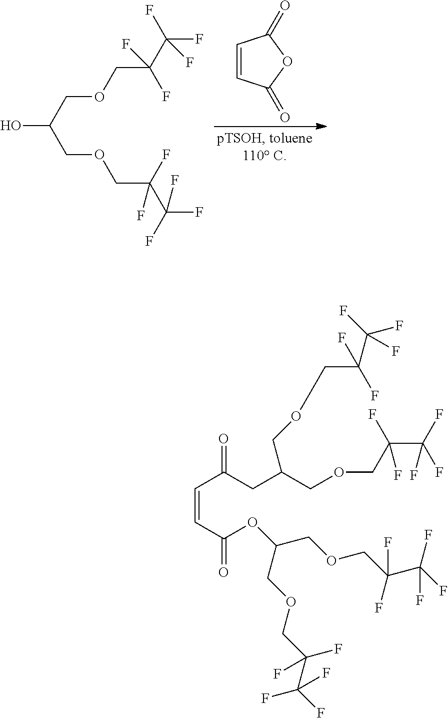

A mixture of 3 eq of 1,3-bis-(2,2,3,3,3-pentafluoropropoxy)propan-2-ol, 1 eq of maleic anhydride and 0.2 eq of toluene-4-sulfonic acid monohydrate in toluene is stirred under reflux for 15 h. The water liberated during the reaction is removed with the aid of the water separator. The reaction is quenched using water. The mixture is subsequently extracted with toluene, and the combined organic phases are washed with DI water, dried over sodium sulfate and filtered. The solvent is distilled off in a rotary evaporator.

Substance: C₂₂H₂₀F₂₀O₈; M=792.37 g/mol

### c) Preparation of the Sulfosuccinate (1A)

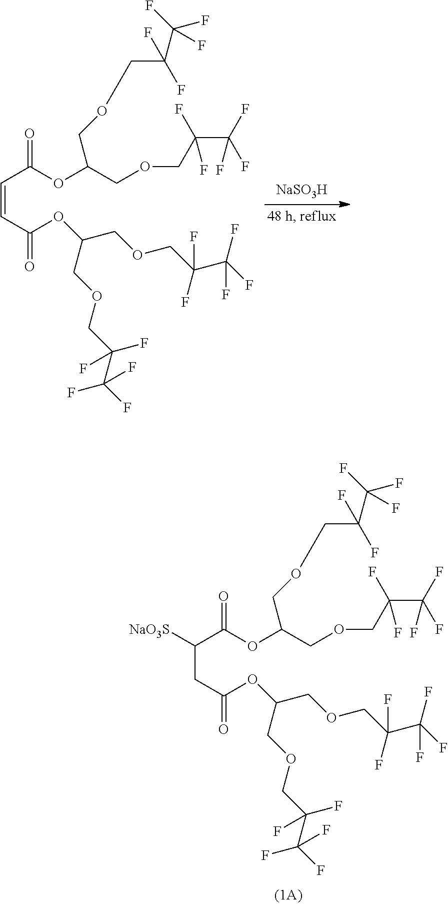

1.5 eq of sodium hydrogensulfite (39% solution in water) are added to a solution of 1 eq of maleic acid ester in 44 ml of 2-propanol at 50° C., and the mixture is stirred under reflux for 48 h. DI water and MTB ether are subsequently added, and the phases are separated. The aqueous phase is extracted with MTB ether, and the combined organic phases are washed with saturated sodium chloride solution and DI water, dried over sodium sulfate and filtered. The solvent is distilled off in a rotary evaporator.

Purification: filtration through Si silica gel.

Eluent toluene/ethyl acetate 1/1

Substance: C₂₂H₂₁F₂₀O₁₁S*Na; M=896.43 g/mol

### Example 2

### Synthesis of the C₂F₅-Functionalised Sulfotricarballylate of the Formula (IB)

### a) Preparation of the Aconitic Acid Ester

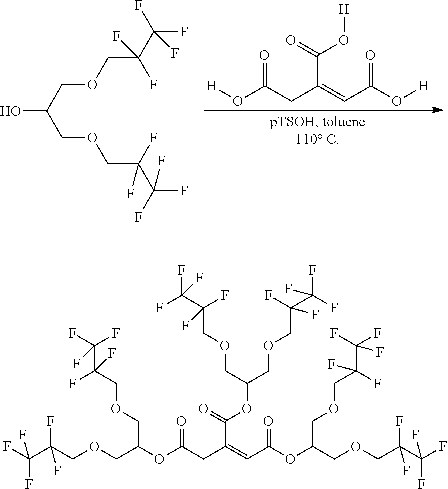 [SMILES: OC(COCC(F)(F)C(F)(F)F)COCC(F)(F)C(F)(F)F ; O=C(O)/C=C(/CC(=O)O)C(=O)O ; O=C(/C=C(/CC(=O)OC(COCC(F)(F)C(F)(F)F)COCC(F)(F)C(F)(F)F)C(=O)OC(COCC(F)(F)C(F)(F)F)COCC(F)(F)C(F)(F)F)OC(COCC(F)(F)C(F)(F)F)COCC(F)(F)C(F)(F)F]

A mixture of 5 eq of 1,3-bis-(2,2,3,3,3-pentafluoropropoxy)propan-2-ol, 1 eq of aconitic acid and 0.2 eq of toluene-4-sulfonic acid monohydrate in toluene is stirred under reflux for 15 h. The water liberated during the reaction is removed with the aid of the water separator. The reaction is quenched using water. The mixture is subsequently extracted with toluene, and the combined organic phases are washed with water, dried over sodium sulfate and filtered. The solvent is distilled off in a rotary evaporator.

Substance: C₃₃H₃₀F₃₀O₁₂; M=1188.55 g/mol

### b) Preparation of the Aconitic Ester Sulfonate (1B)

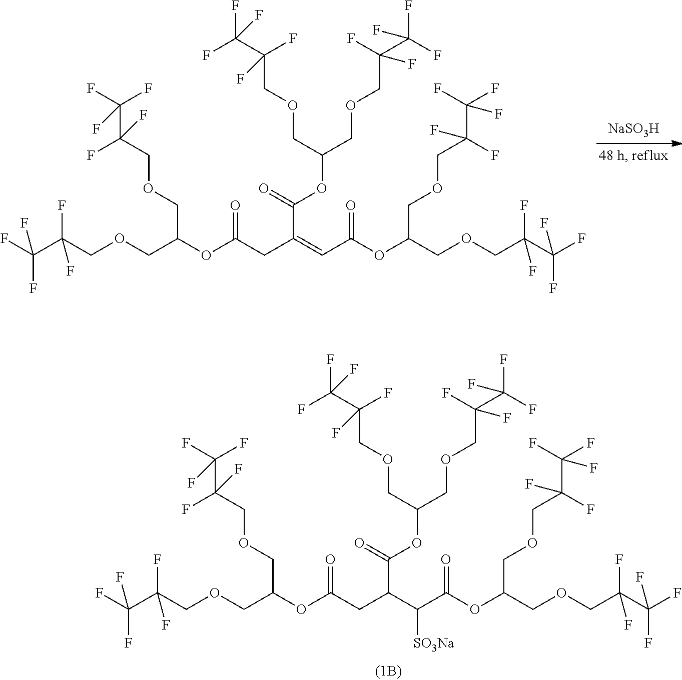

1.5 eq of sodium hydrogensulfite (39% solution in water) are added to a solution of 1 eq of the triester in 44 ml of 2-propanol at 50° C., and the mixture is stirred under reflux for 48 h. DI water and MTB ether are subsequently added, and the phases are separated. The aqueous phase is extracted with MTB ether, and the combined organic phases are washed with saturated sodium chloride solution and DI water, dried over sodium sulfate and filtered. The solvent is distilled off in a rotary evaporator.

Purification: filtration through Si silica gel.

Eluent toluene/ethyl acetate 1/1

Substance: C₃₃H₃₁F₃₀O₁₅S*Na; M=1292.61 g/mol

### Example 3

### Determination of the Static Surface Tension

The static surface tensions γ of aqueous surfactant solutions having various concentrations c (percent by weight) are determined.

Instrument: Dataphysics tensiometer (model DCAT 11)

Temperature of the measurement solutions: 20°±0.2° C.

Measurement method employed: measurement of the surface tension using the Wilhelmy plate method.

Plate: platinum, length=19.9 mm

In the plate method, the surfaces or interface tension of the surfactant solution is calculated from the force acting on the plate length wetted, in accordance with the following formula.

$$γ = \frac{F}{L \cdot cos θ} = \frac{F}{L}$$

γ=interfacial or surface tension; F=force acting on the balance; L=wetted length (19.9 mm); θ=contact angle)

The plate consists of roughened platinum and is thus optimally wetted so that the contact angle θ is close to 0°. The term cos θ therefore approximately reaches the value 1, so that only the measured force and the length of the plate have to be taken into account.

The measurement values for the sulfosuccinate according to Example 1c) are reproduced in Table 1. FIG. 1 shows the static surface tension as a function of the concentration for the sulfosuccinate according to Example 1c).

TABLE 1

| c [g/l] | γ [mN/m] |
|---|---|
| 0.0012 | 48.48 |
| 0.0015 | 47.685 |
| 0.0018 | 46.485 |
| 0.0023 | 45.699 |
| 0.0028 | 44.383 |
| 0.0035 | 43.517 |
| 0.0043 | 42.179 |
| 0.0054 | 40.983 |
| 0.0066 | 39.913 |
| 0.0082 | 38.323 |
| 0.0095 | 36.339 |
| 0.0118 | 34.927 |
| 0.0147 | 34.011 |
| 0.0182 | 32.426 |
| 0.0226 | 31.047 |
| 0.0280 | 29.224 |
| 0.0348 | 28.142 |
| 0.0431 | 27.201 |
| 0.054 | 25.905 |
| 0.066 | 25.165 |
| 0.082 | 24.18 |
| 0.102 | 23.191 |
| 0.127 | 22.506 |
| 0.157 | 21.797 |
| 0.195 | 21.145 |
| 0.242 | 20.746 |
| 0.3 | 20.54 |
| 1 | 20.36 |

### Example 4

### Determination of the Dynamic Surface Tension

The dynamic surface tension γ of a 0.1% (percent by weight) aqueous solution of the compound to be investigated is determined.

Measurement method employed: measurement of the surface tension using the bubble pressure method

Instrument: SITA tensiometer (model t 60)

Temperature of the measurement solutions: 20° C.±0.2° C.

In the measurement of the dynamic surface tension, air bubbles are forced through a capillary into the surfactant solution at different rates. From the resultant pressure change, the surface tension can be determined as a function of the bubble life using the following equation:

$$γ = \frac{r ( {p}_{max} - ρ \cdot g \cdot h )}{2}$$

Pₘₐₓ=maximum pressure, ρ=density of the liquid, h=immersion depth, r=radius of the capillary

The measurement values for the sulfosuccinate according to Example 1c) are shown in Table 2. FIG. 2 shows the dynamic surface tension as a function of the bubble lifetime for the sulfosuccinate according to Example 1c).

TABLE 2

| Bubble lifetime [ms] | γ [mN/m] |
|---|---|
| 31 | 67.6 |
| 38 | 64.8 |
| 51 | 60 |
| 65 | 54.9 |
| 86 | 47.6 |
| 110 | 40.7 |
| 147 | 35.2 |
| 188 | 31.9 |
| 243 | 29.5 |
| 315 | 27.7 |
| 412 | 26.7 |
| 544 | 25.6 |
| 720 | 24.6 |
| 891 | 23.9 |
| 1266 | 23 |
| 1678 | 22.3 |
| 2213 | 21.9 |
| 2470 | 21.8 |
| 3208 | 21.2 |
| 3866 | 21 |
| 5442 | 20.3 |
| 6876 | 19.9 |
| 9092 | 19.4 |
| 11533 | 19.2 |
| 16194 | 18.9 |
| 19523 | 18.9 |
| 25930 | 18.7 |
| 30962 | 18.8 |
| 46049 | 18.7 |
| 54289 | 18.6 |

### BRIEF DESCRIPTION OF DRAWINGS

FIG. 1 shows the static surface tension as a function of the concentration for the sulfosuccinate according to Example 1c).

FIG. 2 shows the dynamic surface tension as a function of the bubble lifetime for the sulfosuccinate according to Example 1c).

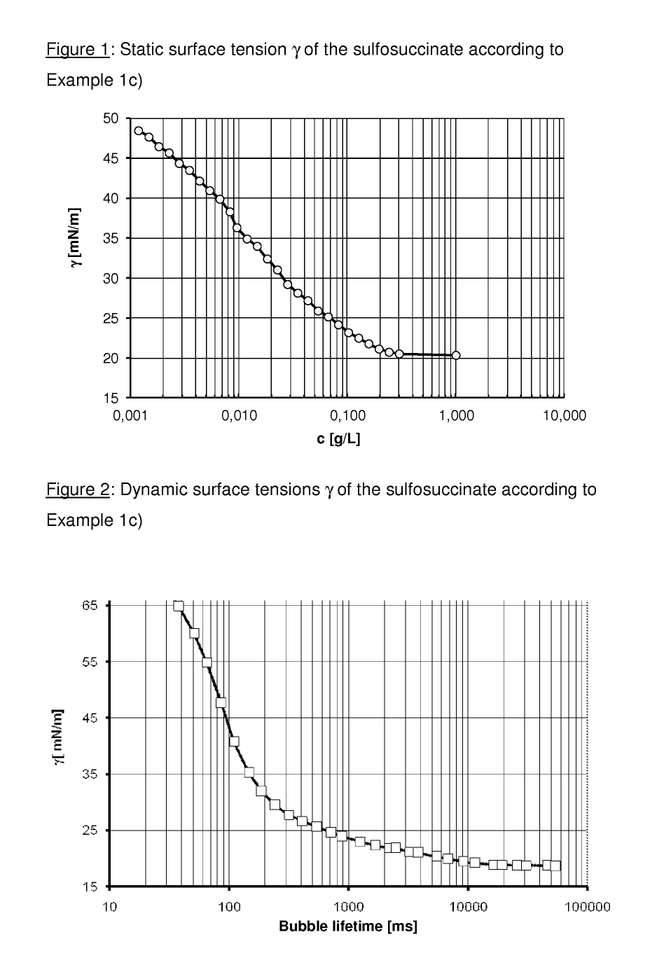

1. Compounds of the formula (I) 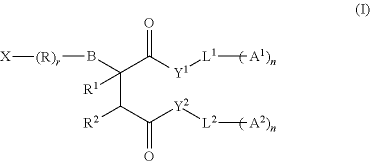
where
X is a hydrophilic group,
R is linear or branched alkylene, where one or more non-adjacent C atoms may be replaced by O, S, and/or N,
r is 0 or 1,
B is a single bond, O, NH, NR′, CH₂, C(O)—O, S, CH₂—O, O—C(O), O—C(O)—O, N—C(O), C(O)—N, O—C(O)—N, N—C(O)—N, SiR′₂—, SiR′₂—O, O—SO₂ or SO₂—O,
where R′ is linear or branched alkyl,
R¹ and R² are, independently of one another, hydrogen or —CH₂—COY³-L³-(A³)ₙ₃,
Y¹, Y² and Y³ are, independently of one another, O, S or N,
L¹, L² and L³, independently of one another, are linear or branched alkylene,
where one or more non-adjacent C atoms may be replaced by O, S, and/or N,
A¹, A² and A³ are, independently of one another, hydrogen or a group of the structure —Zⁱ(CR³R⁴)ₘᵢRfⁱ,
i is 1, 2 or 3,
Zⁱ is O, S or N,
R³ and R⁴ are, independently of one another, hydrogen or an alkyl group,
Rfⁱ is a fluorine-containing radical,
n1, n2 and n3 are, independently of one another, 1-6,
m1, m2 and m3 are, independently of one another, 0-5
and the compounds contain at least one Rfⁱ group.

2. Compounds according to claim 1, characterised in that at least four, preferably four, six or nine, Rfⁱ group are present.

3. Compounds according to claim 1, characterised in that Y¹, Y² and Y³ are equal to O.

4. Compounds according to claim 1, characterised in that L¹=L²=L³ and are equal to linear or branched alkyl having 1 to 10 C atoms.

5. Compounds according to claim 1, characterised in that the fluorinated groups Rfⁱ used are branched or unbranched, perfluorinated alkyl radicals having 1 to 10 C atoms, preferably 1 to 6 C atoms, in particular 1-4 C atoms.

6. Compounds according to claim 1, characterised in that X is an anionic group, preferably —SO₃⁻, —OSO₃⁻, —PO₃²⁻, or OPO₃²⁻, in particular —SO₃⁻.

7. Compounds according to claim 1, characterised in that X is a cationic group, preferably —NR¹R²R³⁺Z—, where R¹, R² and R³ each stand, independently of one another, for H, C₁₋₃₀-alkyl, Ar or —CH₂Ar and Ar stands for an unsubstituted or mono- or polysubstituted aromatic ring or condensed ring systems having 6 to 18 C atoms in which, in addition, one or two CH groups may be replaced by N.

8. Compounds according to claim 1, characterised in that X is a nonionic group, preferably linear or branched alkyl, where one or more non-adjacent C atoms may be replaced by O, S and/or N, —OH, —OCOCR═CH₂ and —O-(glycoside)ₒ, wherein o stands for an integer from 1 to 10.

9. Compounds according to claim 8, characterised in that X equal to R—(O—CH₂CHR)ₘ— where m=an integer from the range from 1 to 100, preferably 1 to 30, and R=H or C₁₋₄-alkyl.

10. Compounds according to claim 1, characterised in that X is an amphoteric group, preferably selected from the functional groups of the acetyldiamines, the N-alkylamino acids, the betaines, the amine oxides or corresponding derivatives.

11. Compounds according to claim 1, characterised in that they conform to the formulae (II), (III) or (IV)

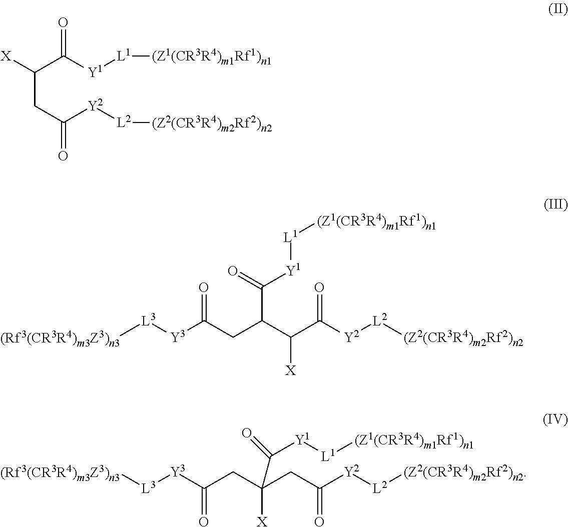

12. Compounds according to claim 11, characterised in that Y¹, Y², Y³, Z¹, Z² and Z³ are equal to O.

13. Compounds according to claim 1, characterised in that the compounds conform to the formula (V) where yi and zi are, independently of one another, equal to 1-10:

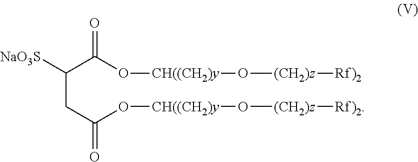

14. Compounds according to claim 1, characterised in that the compounds conform to the formula (VI) where yi and zi, are independently of one another, equal to 1-10:

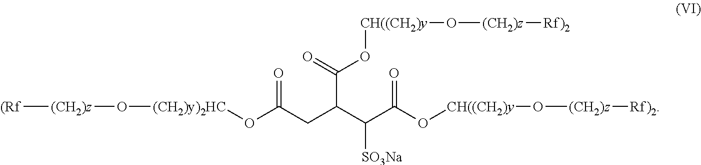

15. A method for reducing the surface tension of a liquid, comprising adding a compound of claim 1 to said liquid.

16. A paint or coating composition or printing ink, comprising a compound of claim 1 and a further compound suitable in a paint or coating composition or printing ink.

17. A method for improving the flow behaviour or wetting ability of a coating formulation, comprising adding a compound of claim 1 to said formulation.
